(19) United States
(12) Patent Application Publication
(10) Pub. No.: US 2026/0122727 A1
(43) Pub. Date: Apr. 30, 2026

(54) HEATING OF TOBACCO IN DISPOSABLE CARTRIDGES WITHOUT COMBUSTION

(71) Applicant: Philip Morris Products S.A., Neuchatel (CH)

(72) Inventor: MARK KRIETZMAN, PALOS VERDES ESTATES, CA (US)

(73) Assignee: Philip Morris Products S.A., Neuchatel (CH)

(21) Appl. No.: 19/006,076

(22) Filed: Dec. 30, 2024

Related U.S. Application Data
(63) Continuation-in-part of application No. 18/927,146, filed on Oct. 25, 2024.
Application No. 18/927,146 is a continuation of application No. 18/592,335, filed on Feb. 29, 2024, now Pat. No. 12,160,935.
Application No. 18/592,335 is a continuation-in-part of application No. 18/133,991, filed on Apr. 12, 2023, now Pat. No. 11,979,949.
Application No. 18/133,991 is a continuation-in-part of application No. 17/211,721, filed on Mar. 24, 2021, now Pat. No. 11,647,566.
Application No. 17/211,721 is a continuation-in-part of application No. 16/118,244, filed on Aug. 30, 2018, now Pat. No. 10,986,872.
Application No. 16/118,244 is a continuation of application No. 15/045,410, filed on Feb. 17, 2016, now Pat. No. 10,076,137.
Application No. 18/133,991 is a continuation-in-part of application No. 17/147,030, filed on Jan. 12, 2021, now Pat. No. 11,770,877.
Application No. 17/147,030 is a continuation of application No. 16/410,858, filed on May 13, 2019, now Pat. No. 10,893,707.
Application No. 16/410,858 is a continuation-in-part of application No. 16/118,244, filed on Aug. 30, 2018, now Pat. No. 10,986,872.
Application No. 16/118,244 is a continuation-in-part of application No. 15/898,629, filed on Feb. 18, 2018, now Pat. No. 10,299,515.
Application No. 15/898,629 is a continuation-in-part of application No. 15/045,442, filed on Feb. 17, 2016, now Pat. No. 9,894,936.
(63) Continuation-in-part of application No. 18/621,058, filed on Mar. 28, 2024, now Pat. No. 12,302,458.
Application No. 18/621,058 is a continuation of application No. 18/412,194, filed on Jan. 12, 2024, now Pat. No. 12,245,337.
Application No. 18/412,194 is a continuation-in-part of application No. 18/133,991, filed on Apr. 12, 2023, now Pat. No. 11,979,949.
(63) Continuation-in-part of application No. 18/412,194, filed on Jan. 12, 2024, now Pat. No. 12,245,337.
(63) Continuation-in-part of application No. 18/233,240, filed on Aug. 11, 2023, now abandoned.
Application No. 18/233,240 is a continuation-in-part of application No. 17/147,030, filed on Jan. 12, 2021, now Pat. No. 11,770,877.
(60) Provisional application No. 62/116,926, filed on Feb. 17, 2015.
(60) Provisional application No. 62/116,926, filed on Feb. 17, 2015.
(60) Provisional application No. 62/116,926, filed on Feb. 17, 2015.
(60) Provisional application No. 62/127,817, filed on Mar. 3, 2015.
(60) Provisional application No. 62/127,817, filed on Mar. 3, 2015.
(60) Provisional application No. 62/127,817, filed on Mar. 3, 2015.
(60) Provisional application No. 62/184,396, filed on Jun. 25, 2015.
(60) Provisional application No. 62/184,396, filed on Jun. 25, 2015.
(60) Provisional application No. 62/184,396, filed on Jun. 25, 2015.
(60) Provisional application No. 62/208,786, filed on Aug. 23, 2015.
(60) Provisional application No. 62/208,786, filed on Aug. 23, 2015.
(60) Provisional application No. 62/208,786, filed on Aug. 23, 2015.
(60) Provisional application No. 62/270,557, filed on Dec. 21, 2015.
(60) Provisional application No. 62/270,557, filed on Dec. 21, 2015.
(60) Provisional application No. 62/270,557, filed on Dec. 21, 2015.
(60) Provisional application No. 62/551,234, filed on Aug. 29, 2017.
(60) Provisional application No. 63/620,181, filed on Jan. 12, 2024.
(60) Provisional application No. 63/621,909, filed on Jan. 17, 2024.

(51) Int. Cl. H05B 1/02; A24B 15/16; A24F 40/40; A24F 40/42; A24F 40/46; A24F 40/50; A24F 40/60; H05B 3/14; H05B 3/42

(52) U.S. Cl. CPC H05B 1/0244; A24B 15/16; A24F 40/40; A24F 40/42; A24F 40/46; A24F 40/50; H05B 1/0225; H05B 3/146; H05B 3/42; A24F 40/60; H05B 2203/014; H05B 2203/021; H05B 2203/022

(57) ABSTRACT

Efficient heating within a receiver of a consumable cartridge containing tobacco (or other plant material) without combustion is disclosed. A disposable consumable cartridge having one or more frangible sections is configured to deform said sections upon insertion into a case with a heating chamber or receiver. The receiver is generally hollow and configured to be in thermal communication with at least one heating element whereby heat is transferred to the interior of the receiver whereby at least one of air in a receiver and material within a consumable is heated.

### RELATED APPLICATIONS

This application claims the benefit of United States (U.S.) Provisional Pat. Application Ser. No. 63/620,181 entitled “VAPORIZING CONSUMABLES HEATED WITH CONVECTION AND CONDUCTION IN A PORTABLE DEVICE” filed Jan. 12, 2024 and U.S. Provisional Pat. Application Ser. No. 63/621,909 entitled “CONSUMABLES HEATED WITH CONVECTION AND CONDUCTION IN A PORTABLE DEVICE” filed Jan. 17, 2024 and the entirety of each application is hereby incorporated by this reference as if fully set forth herein.

This application is a Continuation In Part of U.S. patent application Ser. No. 18/927,146 entitled “HEATING WITHOUT BURNING TOBACCO, IN A DISPOSABLE CONSUMABLE, FOR INHALATION” filed Oct. 25, 2024, and is a Continuation of U.S. Pat. No. 12,160,935 (also Ser. No. 18/592,335) entitled “VAPORIZING CONSUMABLES HEATED WITH CONVECTION AND CONDUCTION IN A PORTABLE DEVICE” filed Feb. 29, 2024. U.S. Pat. No. 12,160,935 is a Continuation In Part of U.S. patent application Ser. No. 18/133,991 (also U.S. Pat. No. 11,979,949) filed Apr. 12, 2023 entitled “PORTABLE MULTIZONE INDUCTION VAPORIZER FOR TOBACCO CONSUMABLES” which is a Continuation of U.S. patent application Ser. No. 17/147,030, (also U.S. Pat. No. 11,707,877) filed Jan. 12, 2021, entitled “PORTABLE TEMPERATURE CONTROLLED AROMATHERAPY VAPORIZERS” which is a Continuation of U.S. Pat. No. 10,893,707, filed May 13, 2019 entitled “Portable Temperature Controlled Aromatherapy Vaporizers” which is a Continuation of U.S. Pat. No. 10,299,515, filed Feb. 18, 2018 entitled “Dynamic Zoned Vaporizer” which claimed the benefit of U.S. Provisional Application Ser. No. 62/551,234 entitled “ZONED VAPORIZERS” filed Aug. 29, 2017 and is a Continuation in part of U.S. Pat. No. 9,894,936, filed Feb. 16, 2016 and entitled “Zoned Vaporizer” U.S. Pat. No. 9,894,936 which claimed the benefit of U.S. Provisional Pat. Application Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications are incorporated by reference herein in their entirety as if fully set forth herein.

U.S. Pat. No. 10,893,707 is also a Continuation In Part of U.S. Pat. No. 10,986,872 (also Ser. No. 16/188,244) entitled “VAPORIZER AND VAPORIZER CARTRIDGES” filed Aug. 30, 2018 which is a Continuation of U.S. Pat. No. 10,076,137 entitled “VAPORIZER AND VAPORIZER” CARTRIDGES filed Feb. 17, 2016 and which claimed the benefit of U.S. Provisional Pat. Application Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications and patents are incorporated by reference herein in their entirety as if fully set forth herein.

U.S. patent application Ser. No. 18/133,991 is also a Continuation In Part of U.S. patent application Ser. No. 17/211,721, filed Mar. 24, 2021, and entitled “VAPORIZERS WITH CARTRIDGES WITH OPEN SIDED CHAMBER” which is a Continuation In Part of U.S. Pat. No. 10,986,872 filed Aug. 30, 2018 entitled “VAPORIZER AND VAPORIZER CARTRIDGES” which is a Continuation of U.S. Pat. No. 10,076,137, filed Feb. 17, 2016 entitled “VAPORIZER AND VAPORIZER CARTRIDGES” which claims the benefit of U.S. Provisional Pat. Application Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications and patents are incorporated by reference herein in their entirety as if fully set forth herein.

This application is a Continuation of U.S. patent application Ser. No. 18/412,194 entitled “VAPORIZING CONSUMABLES HEATED WITH CONVECTION AND CONDUCTION IN A PORTABLE DEVICE” filed Jan. 12, 2024 which is a Continuation in Part of U.S. patent application Ser. No. 18/133,991 (also U.S. Pat. No. 11,979,949) filed Apr. 12, 2023 entitled “PORTABLE MULTIZONE INDUCTION VAPORIZER FOR TOBACCO CONSUMABLES” the disclosures of each of the above referenced applications and patents are incorporated by reference herein in their entirety as if fully set forth herein.

This application is a Continuation of U.S. patent application Ser. No. 18/621,058 entitled “PORTABLE NON-COMBUSTION VAPORIZER FOR TOBACCO CONSUMABLES” filed Mar. 28, 2024, which is a Continuation In Part of U.S. patent application Ser. No. 18/412,194 the disclosures of each of the above referenced applications and patents are incorporated by reference herein in their entirety as if fully set forth herein.

This application is a Continuation In Part of U.S. patent application Ser. No. 18/233,240 entitled CONTROLLED HEATING OF PLANT MATERIAL IN DISPOSABLE CARTRIDGES WITHOUT COMBUSTION″ filed Aug. 11, 2023 which claimed the benefit of U.S. Provisional Pat. Application Ser. No. 63/437,698 entitled “VAPORIZATION CARTRIDGE ZONED HEATING” filed Jan. 8, 2023 and is also a Continuation In Part of U.S. Pat. No. 11,707,877 filed Jan. 12, 2021 the disclosures of each of the above referenced applications and patents are incorporated by reference herein in their entirety as if fully set forth herein.

### BACKGROUND OF THE DISCLOSURE

### Field of the Disclosure

The present disclosure relates generally to smokeless disposable cartridges containing vaporizable oils, compounds and/or other plant-based material which upon appropriate heating controllably releases vapor without combustion.

### Related Art

Vaporizer for plant-based materials and/or essential oils is known. Vaporizers which allow a fluid gas containing the vapor and other residues to follow a fluid pathway from source of vapor to user inhalation exist. Cannabis, hemp, tobacco and other botanicals have been known in the art to be vaporized or burned to release organic material in the form of inhalable material. Vaporizing at correct temperatures can boil off the oils for inhalation without combusting the plant material.

Vaporizer for plant-based materials and essential oils and exist. Vaporizers allow aromatherapy or inhalation. Vaporizers which allow inhalation from a fluid pathway whereby gas containing the vapor to heat not burn without combustion by products through a fluid pathway from source of vapor to exists. Herbs and botanicals have been known in the art to be vaporized or burned to release organic material in the form of inhalable material.

Lavender vaporizes at 260° F. Tobacco vaporizes between 257° F. to over 392° F. Green tea vaporizes between about 175° C. to over 185° C. Valerian vaporizes at about 235° C. Chamomile used to aid in the relief of anxiety vaporizes at about 380° F. Peppermint vaporizes at about 255° F. Peppermint is also known to ease symptoms of allergies and asthma, in addition to alleviating some of the side effects that come along with the common cold or a sinus infection. Cannabis, has a range at which it can be heated to release different cannabinoids as vapor without burning the organic material from below 200 F to about 430 F.]

Heating a cartridge configured to contain organic plant material and/or infused oils on a carrier material may, in some instances, overheat at least portions thereof and therefore combust, overheat or otherwise release unwanted substance which may include carcinogens and chemicals into the vapor.

It is therefore a desideratum to have a consumable suitable for heating device wherein heating the consumable avoids combustion and/or heat is better managed.

### DESCRIPTION

A consumable configured for heating without combustion. A method, system and device is disclosed which can heat not burn plant material such as tobacco in a disposable cartridge (consumable) through a fluid pathway.

Aspects of the devices, systems and methods disclosed herein include a consumable cartridges and vaporizer for heating without combustion of volatile compounds from plant material in a cartridge. A heating device with a controller, a power supply in signal communication with the controller, a heater with at least one heating element affixed cross-sectionally around a portion of an outer wall of a generally tubular receiver having an open end, wherein each heating element is controlled via power allocated by the controller and at least one temperature sensor in thermal communication with the wall of the receiver and in signal communication with the controller; and a cartridge having an inhalation end and a containment portion configured to fit within the receiver and be in thermal communication with the at least one heating element with at least tobacco material within the containment portion. An aliquot of air resides within the receiver between the inner wall and the cartridge. The receiver is also a heat transfer body configured to pass heat from the heater, when the device is “on” to at least the air within the receiver and the containment portion. Air can be replenished in the receiver by way of a fluid pathway configured for air to enter the receiver. The controller is configured to receive a temperature sensor input and control. a timing and/or an amount of power applied to activate the at least one heating element. The fluid pathway is at least one of an air intake and the open end of the receiver.

In some instances multiple conductive regions within the inner wall of the containment portion. In some instances the conductive regions in the containment portion are separated from each other by non-conductive areas of the inner wall of the containment portion. In some instances the plant material includes at least two of hemp, cannabis and tobacco.

Aspects of the devices, systems and methods disclosed herein for a consumable cartridges and vaporizer heating without combustion of volatile compounds from plant material in a cartridge including placing one or more heating elements affixed cross sectionally as rings around an outside wall of a receiver configured to contain air and receive the disposable cartridge and placing at least one temperature sensor in thermal communication with at least one of the outside wall and an inner wall of the receiver and inserting the disposable cartridge inside the receiver. The disposable cartridge has an inhalation end extending beyond the receiver and a containment end holding material to vaporize within the receiver and the receiver is configured as a heat transfer body to transfer heat from the one or more heating elements to at least a portion of the air in the receiver between the disposable cartridge and the inner wall and to the containment end of the disposable cartridge. In some instances forming signal communications between the one or more heating elements, the at least one temperature sensor and the controller. In some instances the controller receives temperature sensor input and controls the timing and/or an amount of power applied to activate a heating element, of the one or more heating elements, to heat the air in the receiver and the containment end to cause vaporization, without combustion, of the material. In some instances the disposable cartridge has at least one thermally conductive region in the containment end. In some instances. the containment end of the disposable cartridge does not burn, or combust at a temperature configured to vaporize plant material after at least one minute of exposure to such temperature. In some instances the containment portion of the cartridge is configured to not burn, or combust at a temperature configured to vaporize plant material after at least five minutes of exposure to such temperature. In some instances the device, method and system include one or more indicator, whereby a status of a system is visually communicated, and the system includes the disposable cartridge and the receiver. In some instances the material is at least one of hemp, cannabis and tobacco. In some instances the controller is configured to switch the heating element based on at least one of a time and a temperature. In some instances at least one thermally conductive region within the inner wall of the containment portion in thermal communication with the inside wall of the receiver, wherein the inside wall is in thermal communication with one or more heating elements.

Aspects of the devices, systems and methods disclosed herein for a consumable cartridges include a disposable cartridge configured to fit within a receiver. Said consumable is disposable, it is configured with as a generally hollow tube formed of an outer wrap having at least one frangible section forming the generally hollow consumable. It has at least a containment end and an inhalation end with material containing at least tobacco within the containment end. The at least one frangible section of the outer wrap is configured to bend inward when passing over an actuator. The material is placed in the containment end and at least one flow through divider is placed within the consumable near the distal boundary and/or the proximal boundary of the containment end. In some instances the at least one flow through divider is placed in the inhalation end. In some instances the at least one flow through divider is placed at the containment end. In some instances of the at least one flow through dividers is placed at the containment end and one is placed between the material and the inhalation end. In some instances the consumable further comprising at least one thermally conductive region within the containment end. In some instances the at least one thermally conductive region is one of metal and foil.

Aspects of the devices, systems and methods disclosed herein for a consumable cartridges and vaporizer heating without combustion including a heat transfer body configured as a tubular receiver with an open top having an annular outside wall and an annular inner wall and at least one heating element fixed around the tubular receiver, A disposable cartridge having a containment end and an inhalation end, wherein the disposable cartridge is configured to be removably placed in the tubular receiver. An aliquot of air within said tubular receiver between the inner annular wall and the cartridge and the containment end contains plant material. During operation said aliquot of air in the receiver is configured to be heated by heat transfer from the annular inner wall and at least one temperature sensor is in thermal communication with the annular outside wall. The at least one heating element and the at least one temperature sensor are configured to be in signal communication with a controller, and the controller is configured to receive a temperature sensor input and control a timing and/or an amount of power applied to activate the at least one heating element to vaporize said plant material, without combustion, during inhalation. In some instances the plant material is at least one of hemp, tobacco and cannabis.

### FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIGS. 1-4F illustrate aspects of cartridges, actuators and heating devices for a heating without combustion system and method.

FIGS. 5A-5B illustrate aspects of cartridge modules and associated heater with multi-zone heating configuration.

FIGS. 6A-6B illustrate aspects of a cartridge module with keyed receiver and vertical heaters.

FIGS. 6C-6D illustrate aspects of a cartridge module with vertical heaters.

FIGS. 7A and 7B are illustrations of another keyed exemplar for heating a consumable.

FIG. 8 illustrates the inner surface of the cartridge and the containment portion with conductive regions.

FIGS. 9A-9C illustrate aspects of another cartridge module and heater.

FIGS. 10A-10C illustrate aspects of a multi-zone heating method and system for a cartridge vaporizer.

FIGS. 10D and 10E illustrate the inner surface of cartridge and the containment portion laid flat.

FIG. 11 illustrates a process for heating a multi-zone system.

FIG. 12 illustrates a process for heating a removable cartridge system.

FIGS. 13A-13H illustrate aspects of vaporizers and positioning of the removable fluid pathway (consumable) remote from the duct (receiver) wall.

All descriptions and callouts in the Figures and all content therein are hereby incorporated by this reference as if fully set forth herein.

### FURTHER DESCRIPTION

Combustion free heating for a disposable consumable cartridge which is an elongated tube formed of one or more materials which wrap at least a material to vaporize therein. The material to vaporize includes tobacco (or other plant material) to release smoke free vapors which is consider healthier for consumer than combustion and burning of substances such as tobacco. Disclosed herein are aspect of the use of a disposable consumable cartridge having one or more deformable frangible sections and said consumable cartridge wrapping may contain metal foil regions. Said consumable with material therein is insertable into a controlled heating device. During or before insertion of the consumable into a receiver, an actuator deforms the frangible section(s) of the consumable. In some instance the deformation forms an air gap in a separation between at least the frangible deformed section of the consumable and the receiver inner wall. The air within the airgap is an aliquot of air or a volume of air which via its location is heated by the transfer of thermal energy (heat) form the inner wall of the receiver. A controller receiving temperature sensor(s) input controls the supply of power to heating elements thereby heating the consumable and air in the gap.

It is appreciated by those skilled in the art that some of the circuits, components, controllers, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs*), time-to-frequency conversations, frequency-to-time conversions, database mapping, signal processing steps, coding, modulations, demodulations, etc. The controller devices and smart devices disclosed herein operate with memory and processors whereby code is executed during processes to transform data, the computing devices run on a processor (such as, for example, controller or other processor that is not shown) which may include a central processing unit (“CPU”), digital signal processor (“DSP”), application specific integrated circuit (“ASIC”), field programmable gate array (“FPGA”), microprocessor, etc. Alternatively, portions DCA devices may also be or include hardware devices such as logic circuitry, a CPU, a DSP, ASIC, FPGA, etc. and may include hardware and software capable of receiving and sending information.

Heating logic turns on/off heating elements forming zones to heat different sections of the cartridge at different times. In some instances the cartridge has limited orientations of insertion to hold it fixed in the heater and unable to rotate about its axis. In some instances the cartridge is marked with a frangible identifier which is broken on insertion to prevent reuse of a spent cartridge. In some instances the cartridge is marked with an identifier that is stored in memory to turn off the heater if the cartridge has already been used.

FIGS. 1, 2, 3 and 4A and 4B show cartridge 1 with two ends, the first end 2 is an inhalation (or intake) end or portion and the second end 3 is a containment (or heating) end or portion. The containment end is generally linear and the material therein has a distal boundary 3′ near the distal end of the consumable and a proximal boundary 3″ abutting the inhalation end. The cartridge is generally tubular and holds material to be heated. During use a flow of air can air passes into the open front 5 to the containment end 3, through the cartridge and then into the inhalation end 2 and finally out through the open back 6. Optionally, a frangible section 7 may be formed on the cartridge whereby it will be deformed during use with a heater. In some instances the deformation may render the cartridge finished an unable to be reused. In some instance an ID 8 which verifies cartridges non-used status may be added to the cartridge. In some instances small perforations 10 may be formed in the containment end 3 to effectuate better heat flow from heating elements. In some instances a filter or flavor filter 12 is placed within the inhalation end 2 whereby vapor inhaled passes. The filter can remove some materials from the vapor and the flavor filter adds an inhalable flavor to the vapor. A flow through divider 15 such as a screen, filter, spacer or coarse filter allows fluid to pass through may be positioned in the cartridge along any position from the containment to the inhalation end. Organic matter 500 is placed within the containment 3 for use of the cartridge. The organic material is a material containing oils or resins (such as, hemp, tobacco and cannabis) which can be released via heating.

The cartridge is formed of an inexpensive disposable material which will not burn or release toxic or harmful fumes at temperatures that are reached by the heater in the device. An outer wrap 4 generally forms a skin or exterior of the cartridge. In generally for many organic materials the temperature of vaporization will be between 320 F to 450 F. The cartridge may be scarred by the heating process as it is disposable. Paper, fibers such as cotton and hemp, metal, foil, plastic, resins, thermoplastics, ceramics, ceramic doped paper, glass, PEEK, and combination thereof may be suitable material for some or all of the cartridge. The cartridge maybe made of different materials for different regions. For example the containment portion 3 is subjected to the greatest heat. The material or materials therein must be suitable to transfer a sufficient portion of the heat applied to its surface through its wall and into the containment portion to thereby cause vapor of the organic material 500 without burning. In some instances the interior annular wall of the containment portion has one or more conductive regions facing the inside.

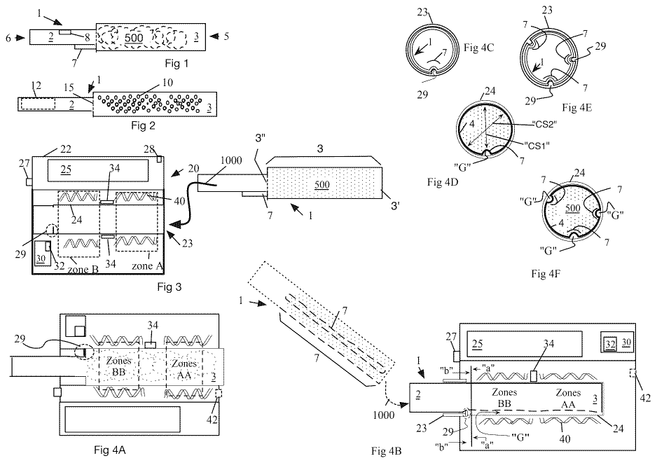

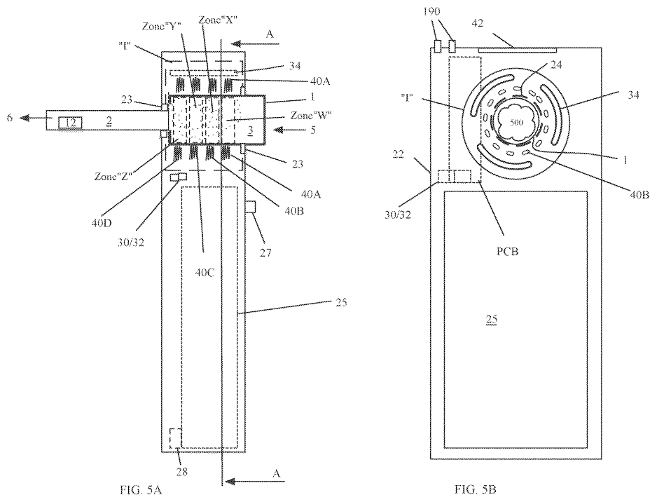

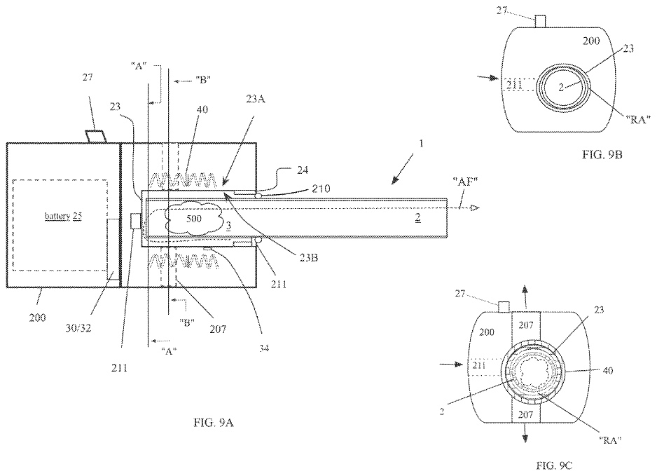

FIGS. 3 and 4D illustrate aspects of a disposable consumable cartridge heated without combustion device. During use the cartridge 1 inserted into a heater 20 via the pathway of arrow 1000. This also may be referred to as a pass-through cartridge device as the cartridge guide is open at both end. The example of the passing the consumable cartridge into the heater is not a limitation and those of ordinary skill in the art will recognize that a non-pass-through configuration is within the scope of this disclosure which is also described in reference to FIGS. 9A-9C. The heater 20 has a case 22 with an interface fluidly connected to a receiver 24 (also referred to as a cartridge guide). The interface 23 opens into a cartridge guide 24. The cartridge guide (receiver) is a channel within the case that is open to allow passage of the cartridge therein. The guide refers to a region within the case that is roughly the cross-sectional circumference or perimeter around the cartridge. Accordingly, if heating elements are placed in the area of the cartridge guide, they would be adjacent to at least a portion of the cartridge. Within the case is a battery 25. A cartridge identification reader 26 may also be added to the case. The identification reader is a sensor that verifies a cartridge is new and has not been previously used. It may determine that a heat indicator has previously been heated, it may determine that a code which is ablated by the heat of use his or is not present thereby interrupting or allowing heating. It may read a code and verify that the code has not been used during a prescribed interval. An on/off switch 27 is shown, and battery may have a charging I/O 28. The case may have a mechanical or electrical mechanical actuator 29 which protrudes in the insertion path of a cartridge and deforms a cartridges The frangible section includes a section of the outer wrap 4 which indents forming a co-axial channel at the frangible section 7 upon insertion. The actuator positioned in or near one of the interface 23 and a receiver 24 (also referred to as a cartridge guide) which are fluidly connected. The deformation of said frangible section may actuate a sensor. Actuation is a communication of the actuator 29 to the controller whereby the controller recognizes the state of the cartridge as “new” and not used and thereby allows electrical current to flow to the heating elements. Within the case is a controller 30. The controller is a microprocessor which may have memory 32 and which controls certain operations of the vaporizer device. Operations may include one or more of time, date, location, security code, on/off, sequence of heating, temperature, indicator display of the heater, battery charging, battery management, battery state of charge indication, communication via illumination, sound, vibration and the like with the user and cartridge verification. Those of ordinary skill in the art will recognize that blue tooth or other wireless or wired connection to a smart phone or computer may also be used to perform some of the controller functions and that would be within the scope of this disclosure. One or more temperature sensors 34 are within the case and near the receiver 24.

The case 22 contains one or more heating elements 40. One or more heater vents 42 may be provided. Both heating elements are shown those of ordinary skill in the art will understand that what is disclosed is one or more zones. In some instance only a single heating zone may be provided, in other instances multiple zones may be utilized and such is within the scope of this disclosure.

In some exemplary implementations a multi-zone heater is disclosed it may have heat zone “A” and “B”. A cartridge, during use, will have corresponding zones “AA”” to “BB” which align generally with the heat zones.

During use one or more zones may be turned on to supply heat, via heating elements, to heat organic material 500 and release vapor. Sequencing the zones for heating is advantageous in that it can reduce power consumption by splitting up the total area to heat into the zones so that less power per zone is needed then power to heat all zones at the same time. Sequencing the zones for heating is also advantageous in that it can release vapor from a discreet amount of organic material at one time thereby leaving less heated or unheated areas of organic material with the same cartridge for a next use. Sequential heating also reduces overheating and supports continuous use while reducing overheating. Overheating, for at least cannabis results in singeing the material which is commonly referred to as a “popcorn” taste.

FIGS. 4B to 4D illustrates aspects of a non-pass-through embodiment of the disposable consumable cartridge heating without combustion device shown in FIG. 4A. During use the cartridge 1 is inserted into a heater 20 via the pathway of arrow 1000. The heater 20 has a case 22 with an interface fluidly connected to a receiver 24 (also referred to as a cartridge guide) open at one end and in fluid communication with the interface 23. The receiver acts as a heating chamber. The cartridge guide (receiver) accepts insertion of the cartridge therein. The cartridge guide refers to a region within the case that is roughly the cross-sectional circumference or perimeter around the cartridge. Accordingly, if heating elements are placed in the area of the cartridge guide, they would be adjacent to at least a portion of the cartridge. Within the case is a protruding mechanical actuator 29 associated with one of the interface and the cartridge guide. The actuator deforms a cartridge's frangible section 7 upon insertion through an interface 23 into a receiver 24 (also referred to as a cartridge guide), the deformation(s) form gaps “G” of air between portions of the cartridge guide and the disposable consumable cartridge. Said air gap(s) “G” contain a volume of air or aliquot before any use. During use the volume of air is replenished by virtue of the negative pressure cause by inhalation on the inhalation end. A fluid communication at the interface forms a fluid pathways(s) for air to pass into the receiver via the interface 23 from the exterior of the device and during an inhalation (negative pressure) be drawn through said consumable from containment end to inhalation end. Additional fluid pathways are formed as one or more areas traversing axially on of the side of the consumable by action of passing over the actuator 29. The frangible section or sections are compressed to form shallow axial strips or regions along their length. The shallow region or regions have cross sectional diameter “CS1” which is less than the cross-sectional diameter of the disposable consumable in areas without shallow regions “CS2” Within the case is a controller 30. The controller is a microprocessor which may have memory 32 and which controls certain operations of the vaporizer device. Operations may include one or more of time, date, location, security code, on/off, sequence of heating, temperature, indicator display of the heater, battery charging, battery management, battery state of charge indication, communication via illumination, sound, vibration and the like with the user. Those of ordinary skill in the art will recognize that blue tooth or other wireless or wired connection to a smart phone or computer may also be used to perform some of the controller functions and that would be within the scope of this disclosure. One or more temperature sensors 34 are within the case and near the receiver 24. FIG. 4C is a cross section along line “a”-“a” of FIG. 4B, a frangible section of the cartridge is deformed via passing over the protruding actuator into the cartridge guide 24. FIG. 4D is a cross section along line “b”-“b” of FIG. 4B, the frangible section of the cartridge is separated from the cartridge guide (also referred to a receiver) inner wall having an indented area in cross section forming an air gap “G” between the receiver wall and indented frangible section. FIG. 4E is an exemplar of FIG. 4C wherein multiple actuators 29 deform the frangible sections 7. FIG. 4F is an exemplar of FIG. 4D having been inserted into the receiver with the multiple actuators whereby the air gaps “G” are surrounding the cartridge.

During use one or more zones may be turned on to supply heat, via heating elements, to heat organic material 500 and release vapor. Areas of the cartridge in contact with the inner wall of the cartridge guide will heat by direct heat transfer from the wall to the cartridge, areas of air near the deformed frangible sections will heat up and be drawn through the cartridge upon inhalation. Sequencing the zones for heating is advantageous in that it can reduce power consumption by splitting up the total area to heat into the zones so that less power per zone is needed then power to heat all zones at the same time. Sequencing the zones for heating is also advantageous in that it can release vapor from a discreet amount of organic material at one time thereby leaving less heated or unheated areas of organic material with the same cartridge for a next use. Sequential heating also reduces overheating and supports continuous use while reducing overheating. Overheating, for at least cannabis results in singeing the material which is commonly referred to as a “popcorn” taste.

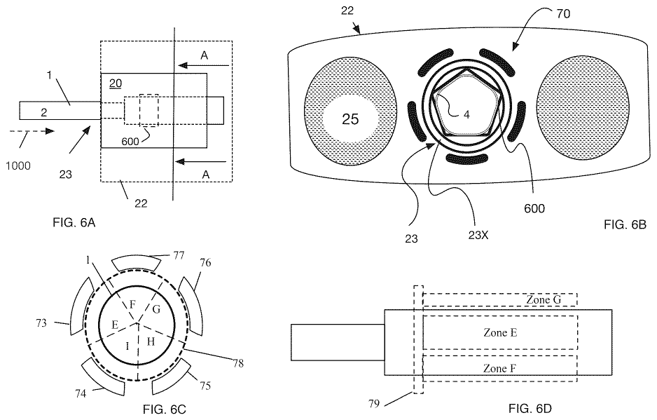

FIGS. 5A-5B show an alternative package for the systems shown in FIGS. 1-3. This disclosure heats the cartridge 1 akin to slicing a sausage. Each of zones “W”-“Z” are sequentially heated. The controller keeps count of which was the last zone to be heated. The controller keeps count of when all zones have been heated and can stop the heat cycle until a spent cartridge is removed and replaced with a new cartridge. It also adds indicators 190. Indicators are shown as LED lights. Indicators are used to communicate status of the device to a user. The communication may be of a spent cartridge, a spent zone that has been heated, the remaining zones to heat, a need for recharge, or remaining zones to heat. Heating elements 40A-4D are in thermal contact with heat zones “W”-“Z”. One or more temperature sensors 34 are within the case and near the receiver 23, each temperature sensor is associate with at least one of a heat zone and heating element. Insulation “I” may be placed around the heater elements inside the case 22. The heater elements may optionally be connected to a PCB board via conductive wires and the controller and memory may also be on that board. The battery 25 is connected via the on/off switch 27 to the controller 30 to supply power to the heat elements. The controller 30 may be connected to separate digital memory 32. The controller initiates each heater element (40A-40D) sequentially to spend each zone and then use the next. When the sequence is complete the device stops heating until reset. All aspects of systems to verify, authenticate and assure that a used cartridge is not reloaded into the device, as described herein with respect to other exemplars are hereby incorporated into this description with respect to FIGS. 5A and 5B.

The cartridge mates with the receiver 23 which places it adjacent to heating elements. The cartridge heating portion (containment) should be constructed so that it does not burn, or combust at exposure temperatures below at least one of 400 degrees F., 410 degrees F., 420 degrees F., 430 degrees F., and 440 degrees F. The failure to burn or combust occurring after at least one of 30 seconds exposure, 1 minute exposure, 2-minute exposure. The failure to burn or combust occurring after at three least 30 second exposures. The failure to burn or combust occurring after at three least 1-minute exposures. The failure to burn or combust occurring after at least four 1 minute's exposures. The failure to burn or combust occurring after at five least 1-minute exposures. The failure to burn or combust occurring after at seven least 1-minute exposures. The failure to burn or combust occurring after at eight least 1-minute exposures

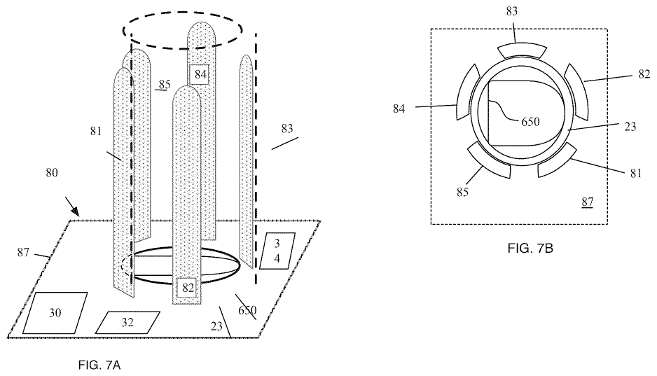

FIGS. 6A-6D shows aspects of another exemplary implementation of the cartridge and heater device. A heater 20 in a case 22 heats a disposable cartridge 1. FIG. 6B is a cut-away view from line “A-A” of FIG. 6A, it shows a receiver 23 and a key guide portion 600 and cartridge 1 looking up the receiver towards the key guide portion 600 remote from the end of the receiver 23X. The key limits cartridge entry to one configuration and prevents rotation about its axis. The cartridge is inserted along the line of arrow 1000 and will partially conform to the key shaped area of the receiver and be shaped. The inserted cartridge will have portions in contact with the receiver wall and portions will be separated by air gaps visible at the 5 corners of the pentagon shaped key guide portion. One or more heating elements are shown. Said heating elements may be a series of vertical heating elements 70 (also referred to as an array). Said elements may be one or more heating elements which wrap at least partially around the receiver (also referred to as outer boundary located between the cartridge and heating elements). Said heating vertical elements are each aligned with a specific region of the heating chamber in an axial direction. In some instances the heater 20 is one or more heating elements encircle at least a part of the receiver (also referred to as outer boundary located between the cartridge and heating elements) and/or heating chamber. The vertical heaters are not a limitations and as disclosed herein heating element(s) at least partially encircling a receiver are within the scope of the disclosure. FIG. 6C is an alternate embodiment without a key guide portion of the aspects of a vertical zone heater 72 showing elements which are aligned with a cartridge 1 and how each element 73-77 forms a corresponding vertical heat zone E-1. An optional boundary 78 (receiver) may be formed between the heater elements 73-77 and the cartridge 1. Accordingly, vertical heat zone “E” is heated by vertical zone heater element 73 and so on. A printed circuit board (PCB) or other support may be used to support the heater elements 73-77. FIG. 6D shows a side view of the cartridge 1 in FIG. 6C with a representation of location of vertical heat zones E-G with a support 79.

The controller supplies power to each one or more heater element in response to temperature sensor data. In some instances the heating may be sequential the turning on/off of a heating element is controlled by the controller. The controller can also receive temperature sensor data to turn one or more heating elements or to maintain a set exposure temperature. The controller can turn on one of the heating elements thereby directing the heat to one heating zone. Memory either volatile or non-volatile will store data on system parameters when the controller is not powered. The controller instructs the on/off of heating elements within the heating array.

FIG. 7A is another exemplary of a vertical heating zone device 80 with heating elements 81-85 affixed to a PCB board or other support 87. A PCB can also support a controller 30 and memory 32 and a temperature sensor 34. Another cartridge orientation key hole 650 is also shown. An optional boundary 78 (also referred to as a receiver) may be formed between the heater elements and the cartridge. FIG. 7B illustrates a top view of the key hole, receiver and heater(s) arrangement of FIG. 7A.

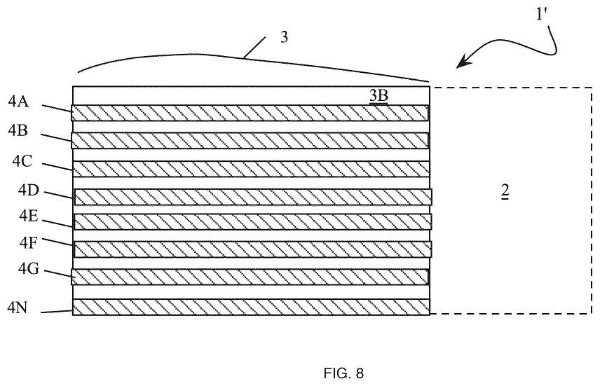

FIG. 8 shows the containment region 3 of the cartridge as a flat sheet l′ before it is assembled into a tubular form. By separating the conductive strips 4A-4N with a gap 4″ of non-conductive (or less conductive) inner wall material the conductive strips are isolated to reduce thermal contact between conductive strips to preventing heating of the entire cartridge at same rate due to the thermal conductance. Isolating the heating elements from all conductive strips via gaps limits a vertical heaters heating to the strip or strips adjacent to the activate heating element. The strips are not aligned one strip per vertical heating element which complicates the system and method by requiring a cartridge to be inserted in an exact position to align heating elements and conductive areas. Gaps limit the thermal contact of strips to isolate heating specific areas. Isolation limits singeing and overheating by allowing an area to cool while another area heats. The plurality of strips and gaps are configured to transfer heat generally from an active heater via closest strips. each other reduces thermal conductance between the conductive strips.

FIGS. 9A-9C show a non-pass through cartridge heating system, device and method. A case 200 and contains the one or more heating elements 40. The cartridge 1 is removable from the case. The cartridge fits into interface 24 whereby the containment end (which is also referred to as distal end) 3 of the cartridge is within the receiver 23 (forming a guide or interface within the heater for the cartridge) which is configured as a heat transfer body thermally communicating heat from the one or more heating elements 40, which include a coil wire, Kapton™ (polyamide) or silicone tape with metalized flat elements, iron-chromium-aluminum (FeCrAl) alloys, nichrome (nickel chrome alloy) wires, filaments or any material which does not outgas at the desired temperatures, are located around the outside annular wall 23A of the receiver 23 to the annular inner wall 23B of the receiver. During use, the air in the receiver “RA” is heated when the heating element heats the annular inner wall and the cartridge inside the receiver. The heated airflow “AF”, during inhalation, flows through the cartridge containment end to the inhalation end 2 (also referred to as the proximal end) extended from the case. An additional seal 210 may be within the case to seal against the cartridge to limit any leakage. An organic material 500 for vaporization is within the distal end near the one or more heater elements 40. One or more vents 207 allow the heater to vent from the case. At least one air intake 211 provides a fluid pathway for air to enter the case and fluidly be drawn into the receiver 23 then through the cartridge from distal end to proximal end and then out for during inhalation. FIG. 9B shows a cut away view of the device along the lone of A-A and FIG. 9C shows a cut-away of the device along the lines of “B-B”.

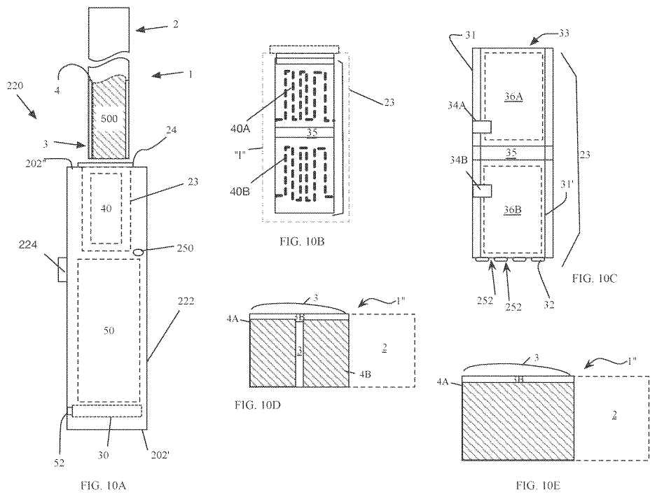

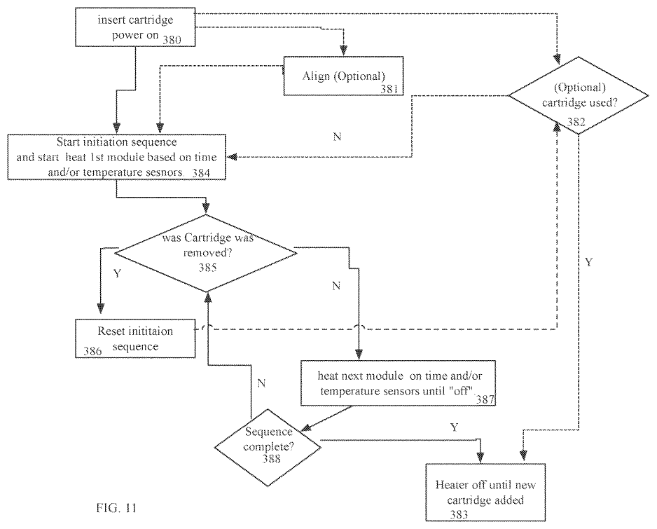

FIGS. 10A-10E discloses aspects of a vaporizer devices 220 comprising an enclosure also referred to as a case 222 which may have one or more user interfaces, such as press or push buttons or switches 224 to communicate with a controller 30. The enclosure is generally hollow it may have a closed bottom 202′ and an open top 202″ A communication display such as illumination via an electroluminescent screen, light emitting diode (LED) or a liquid crystal display may be added, or communication with user may be via sound, or vibration. The cartridge interface 24 forms a passage into the case through the open top and is fluidly connected to a receiver 23. The receiver is configured to receive a cartridge 1 and transfer heat to the containment portion 3 of the cartridge. The cartridge maybe made of different materials for different regions, layers or areas. For example the containment portion 3 is subjected to the greatest heat. The conductive material 4 shown is suitable to transfer sufficient heat applied to the containment portion by the device to thereby cause vapor of the organic material 500 within to heat and vaporize material without burning. The receiver is configured with an outer annular wall 31, an inner annular wall 31′, a floor 32 and an open top 33 which is in thermal communication with a heater system 40. In the heater system 40 there are subzones which are independently controlled by the controller whereby heat is generated via the use of electrical power. Resistance type heaters which may be one or more of against, around or within materials such as ceramic walls, metal or the like and are examples of heating elements. Subzone heating elements 40A-40B are in thermal contact with and wrap around one of the outer annular wall 31 and the inner annular wall 31′ of each zone 36A and 36B to selectively apply heat as directed by the controller. A battery power supply 50 provides electricity to this portable device for functions of the controller, temperature sensors 34A and 34B, heating elements and communications. Communications with user may be provided whereby a user can obtain status of the device or adjust settings. The battery power supply is at least one of rechargeable and replaceable. The heating elements are configured to receive electrical power. The controller is configured to control the supply of electrical power to the heating elements and that control may be via pulse width modulation. Insulation “I” may be added around the receiver chamber and heater system 40. Within the receiver an insulation divider 35 is interposed cross sectionally as a ring between zones to thermally separate at least a portion of zone 36A and 36B. In some instances the insulator ring is between two cylindrical portions or units forming the heater system 40. The insulator divider separates heating zone 36A and 36B. The insulation divider 35 reduces thermal communication between zones by dividing thermal contact between portions of the annular wall to limit heating of the portion of the annular wall not being heated by a heating element. Divider may also be insulator to reduce thermal contact between material 500 in the cartridge containment 3 remote from the active heating element. Although two heating elements are shown those of ordinary skill in the art will understand that what is disclosed is one or more zones. In some instance only a single heating zone may be provided, in other instances multiple zones may be utilized to localize heat zones and such is within the scope of this disclosure.

A recharge connection 52 communicates through the enclosure or case for recharging the battery, it may be a USB or other power connection. Inside the case is a controller 30, optional I/O 52 may be a USB connector (or other standard such as USB-C, micro USB and the like) which may also provide recharging functions and data input/output. Additional memory via solid state device may be provided. In some instances an optional wireless connection via Wi-Fi or Bluetooth or the like may be provided on the appropriate solid state device.

At least one temperature sensor 34, for the chamber or in other instances one temperature sensor 34 per zone 36A and 36B. (a first and a second sensor) such as a thermistor or thermocouple is placed in close proximity to the heater system to communicate data to the controller whereby the energy provided to the heater system and subzones is modulated to maintain a selected temperature. Wireless connection allows connecting the device to a smart phone which can have software (applications) which pair with the device and adjust operation of the device via the controller.

Inside the receiver is a floor that seals the bottom forming an open chamber with a partially blocked floor and the floor provides a fluid pathway for air to be drawn into the chamber. The case 222 provides a series of vents 250 which provide fluid communication from the outside of the case to the inside. A series of intakes 252 create a fluid pathway into the receiver 23 allow air to be drawn through. The material forming the containment portion of the cartridge 1″ is shown laid flat in FIGS. 10D and 10E.

In FIG. 10D two conductive regions 4′ and 4″ are formed on the inner wall 3B of the cartridge 1″ and separated by a less or non-conductive region 3′. The conductive regions are configured to align with heating zone 36A and 36B when the cartridge is assembled, filled with material in the containment and inserted into the receiver 23. Thereby facilitating heating of a zone in the cartridge with the heating element of that zone. Temperature sensors associated with each heating zone provide input to the controller.

The material forming the containment portion of the cartridge 1″ on another exemplar is shown laid flat in FIG. 10E and the one conductive regions 4 is formed on the inner wall 3B of the cartridge 1″ and separated by a less or non-conductive region 3′. The conductive region is configured to align with heating zone 36A and 36B when the cartridge is assembled, filled with material in the containment and inserted into the receiver 23. Thereby facilitating heating of a zone in the cartridge with the heating element of that zone. Temperature sensors associated with each heating zone provide input to the controller

Methods disclosed include a controller that manages heating of a zone at a selected exposure temperatures (SET) to vaporize a portion of the material in the containment area in the accordance with one of variable, preselected and fixed times. The heating of all heating elements may also be referred to as a cycle or a heating cycle. When a cycle is over the cycle has timed out. Temperature sensors are utilized to measure when the chamber or subzone has reached a target temperature. If the amount of time a specific heating element is to be heated is reached the heating of that element has timed out. The controller can track, monitor, measure or otherwise count that heating time. In other instances the controller may switch between subzones, preferably using a PWM protocol to supply power to each heating element separately to maintain a temperature at a predetermined range. Selective heating allows the “off” zone to cool while the “on” zone is heating. Said cooling is effective to reduce singeing and/or combustion of material.

In some instances the controller prohibits heating when a zone has already been heated for a predetermined timeframe. In some instances the controller may accept a user override to allow reheating of a zone or to heat multiple zones simultaneously.

FIG. 11 illustrates aspects of a control sequence and sequence of operation of one or more exemplary implementations disclosed herein. First a cartridge is inserted into a heater unit and the on/off switch is depressed 380. Optionally, a mechanical, optical, or electro-mechanical fixture limits the orientation of the cartridge to a predefined one 381. Optionally, a sensor collects data on the cartridge to determine if it is used 382. If used 383 keep heater off until a new cartridge is added. If cartridge is not used then start power initiation and heat 1.sup.st heating element/module 384 based on at least one of time and/or temperature, until user selects “off” or controller shuts off which may be due to time being completed. Then determine if cartridge has been removed 385. If the cartridge has been removed 386 then rest sequence of operation. If not removed 387 heat next heating element/module until user or controller shuts off. Next determine if sequence of heating is complete 388. If no heat cartridge if it has not been removed 385, else reset 386. If cartridge heating sequence is complete (all heating zones have been heated) then keep heater off until a new cartridge is provided 383.

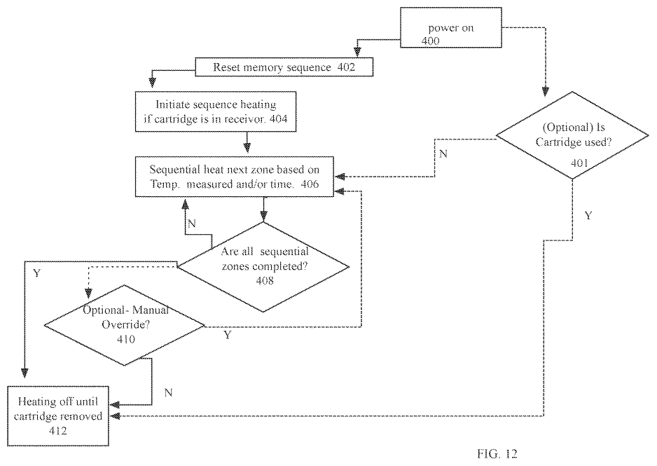

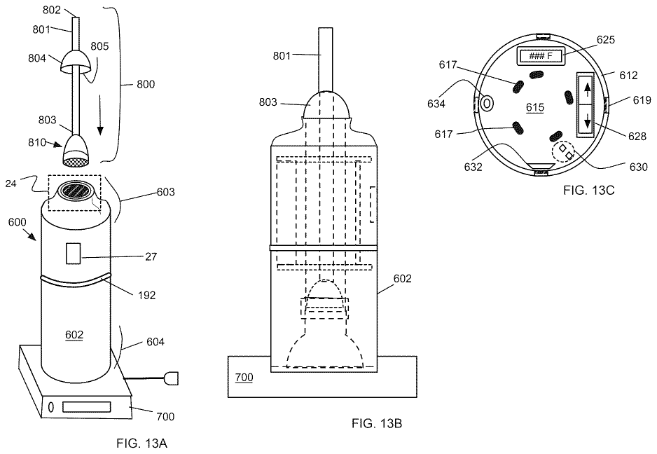

FIG. 12 illustrates aspects of a control sequence and sequence of operation of one or more exemplary implementations disclosed herein. First power is turned on 400. Optionally the system checks if cartridge in receiver is used 401. If used turn heater off until used cartridge is removed 412. After power on the controller resets memory sequence for sequentially heating back to beginning 402. Next the controller initiates sequential heating of zones if a cartridge is in the receiver 404. The controller, which is configured to control sequential heating, controls the application of power to the selected “on” heating elements until changing to the next heating element in the selected sequence 406, based on at least one of temperature during heating of the zone and time. The controller decides if sequential heating is complete 408. If “no” the controller continues the system's sequential heating 406. If “yes”, optionally the controller checks if manual override 410 is selected which continues the sequential heating loop. Otherwise, the controller stops heating any elements until the cartridge is removed 412.

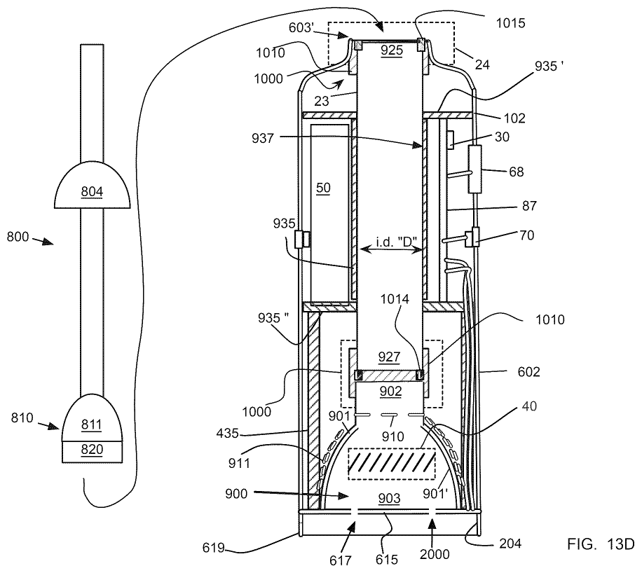

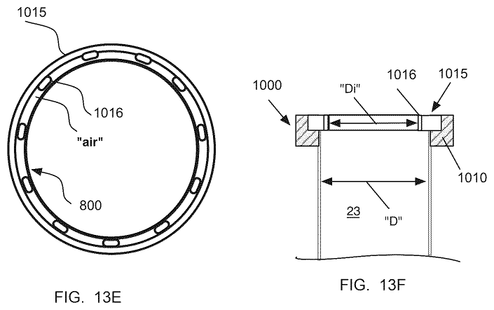

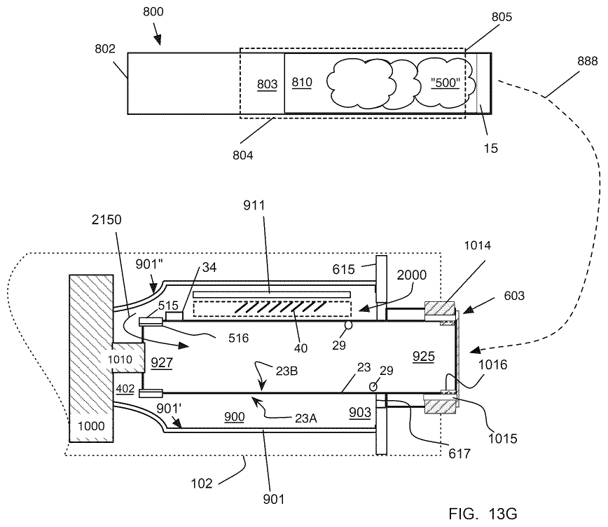

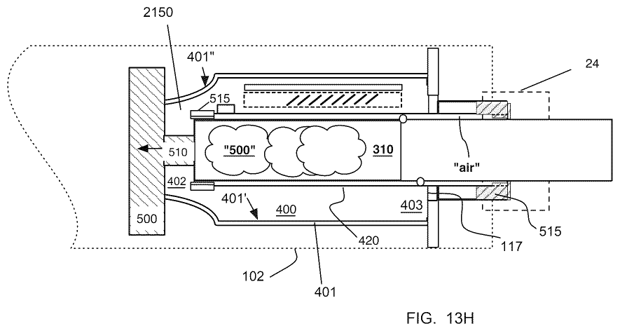

FIGS. 13A-13H show aspects of vaporizers and positioning of the removable fluid pathway (consumable) remote from the duct (receiver) wall. FIG. 13A-13H show aspects of vaporizers which use, at least in part, heated air to vaporize material 500 in a consumable 800 also referred to as a removable fluid pathway (RFP). The external body 602 forms a vessel which is a substantially hollow shroud/cover over a heating system, power supply mechanical components and electronic components. The body has a top region 603 with a top opening which forms a fluid connection from the exterior of the device to the interior. The fluid connect may be to a furnace directly or connects to a duct also called a receiver 24. The body has a bottom region 604 and a bottom edge 612. An interface 24 formed at the top region 603 provides a fluid connection from the exterior of the vessel through the top region 603 to the interior of the body 602 into at the receiver 23. An on/off switch 27 that may be a touch switch, contact or pressure switch is user accessible from the exterior of the body 602. The switch may be push on push off, the switch may be programmable, or controlled by the control board wherein activation causes the device to enter a steady on state to continually heat botanicals for aromatherapy over a preselected period of time. One or more indicators for illumination communications 192 are provided on the body to be visible from the exterior of the body. The illumination includes but is not limited to one or more lenses, LEDs (light emitting diode), electroluminescent band, and may be a series of drilled holes or very thin body areas that an illumination from the LEDs able to penetrate there through. The vessel 600 is shown removable connected to a charging base 700 and a consumable (removable fluid pathway “RFP”) 800. The consumable may material chamber 810 is a containment for material 500, it may be single part or multipart having a bottom 820 and a shaped top 811. In some instances (see FIGS. 13E and 13F) it may be generally tubular with a generally consistent diameter of the consumable.

The consumable (RFP) fits into the receiver 23 which is an open duct like structure having an interface 24 configured to allow insertion of a consumable therein and form a fluid path for both heated air and vapor liberated from material 500. Those of ordinary skill in the art will recognize that a non-removable fluid pathway is within the scope of this disclosure and a chamber with an affixed or non-removable fluid pathway (NRFP) is within the scope. Colored illumination such as light emitting diodes (LEDS) are useful for communications. An illumination visual language is used wherein the one or more printed circuit boards (PCB) “board” 87 with a controller 30, memory 32 and other components to support signal communication and input/output to control functions of the device. PCBs and controllers are well known in the art. Pulse width modulation (PWM) power management, temperature sensor inputs, memory, clock, and Wi-Fi connect ability are a non-exclusive list of PCB “board” components and functions. all be control one or more of color, strobe, frequency, intensity and movement (by turning some LEDs off in the band of lighting) of illumination to convey state of the device. For example, green may mean at temperature and ready to use. Red may mean heating up. Flashing red may mean time to recharge. Blue may mean standby mode.

At the bottom 612 of the body is an inserted, affixed or otherwise attached closure 615 which also may be a part of an internal chassis 935. One or more air intakes 617 may be formed on that closure to provide a fluid passage for external air to be drawn into the vessel during heating and use. Intake vents 619 may also be added to allow air flow through the side edge of the body.

User interface display 625 and inputs 628, recharge to base connectors 630, data/power interface 632 and/or power jack 634 are shown on the bottom closure or floor 615. Inside the body is the heart of control systems and heating and not burning material in consumables. A substantially tubular furnace 900 has a thin wall 901 with an interior surface 401′ forming a container which allows for intake of external air and for air heated therein to exit. In this exemplar the furnace has a narrower diameter first end or open top 902 and a wider diameter second end or bottom 903. Preferably the wall is less than 1 millimeter thick, more preferably less than 0.5 millimeters thick and most preferably less than 0.25 millimeters thick. Suitable materials should have no harmful levels of outgassing at temperatures the furnace will be used at. These materials include but are not limited to phenolic resins, aluminum, titanium, stainless steel, and ceramic. A heating element 905 such as a polyamide, silicon with wire, kanthal or nichrome coiled wire is within the furnace. Optionally insulation 911 may wrap at least some of the thin wall of the furnace.

In some instances a gasket 1000 is configured to form a fluid connection to the material chamber 810 and may also be referred to a chamber gasket interface (CGI) and may form a portion of a guide pathway whereby the material chamber 810 (which may be multipart including a cup bottom 820 and a shaped top 811) fits into the CGI to mate within the receiver 23 the gasket 1000 may be at the region between the receiver and furnace or directly between the furnace and a top mounted chamber. The receiver has an internal diameter (i.d.) denoted by “D”. The receiver has an open top or proximal end 925 and an open bottom or distal end 927 to fluidly connected heat from the furnace. The distal end of the receiver 927 is shown near the first end 902 of a furnace via and the fluid connection is via an insulation member 1010.

The insulation member may be a pliable or semi pliable gasket, silicon tape, molded ring, ceramic, polyimide film or the like and it functions to hold the duct and furnace ends aligned while separating the two ends to limit heat transfer, parasitic losses due to heat transfer. Further the insulating member may be formed to hold and isolate the inserted removable fluid pathway (consumable) 800 from thermal contact with the receiver (see FIGS. 13E and 13F).

A gasket 1000 connects the receiver 23 to the body 602. An insulation member 1010 may be configured as an interface gasket (see FIG. 13D). The interface gasket connects the exterior of the body near the top section 603 and the exterior of the top 925 of the receiver. A band 1015 may be used to separate the receiver and the body from direct physical contact and to limit thermal contact via the insulating properties of the insulation member 1010. The band has an internal diameter “Di” which is less than the internal diameter “D” of the receiver. At least the band is compressible. The insulation member is preferably compressible. The band (1015) may be a homogeneous thickness or it may be non-homogeneous having thicker portions (1016) leaving spaces between the edge of the cartridge (RFP) and the band. Within those space is “air”. The band or band and insulation member combination should allow the press fit of the cartridge into the receiver without the cartridge touching the receiver internal wall 23B wall.

The vessel 600 contains a power supply such as lithium ion batteries 50 and it can be charged with one or more of the recharge-to-base connectors 630, data/power interface 632 and/or power jack 634. Accordingly, it may be charged on or off the base.

The removable fluid pathway 800 provides a substantially hollow flow channel 801 which has an outlet 802 for inhalation and an inlet 803 in fluid communication with the material chamber 810. Optionally a spacer 804 may be fitted to the exterior of the flow channel to one or more of act as a heat exchanger to the flow channel, position the fluid pathway 800 within the receiver 23, provide a grab for a user to remove the fluid pathway 800.

A heating system 40 such as coil wire, Kapton™ (polyamide) or silicone tape with metalized flat elements, iron-chromium-aluminum (FeCrAl) alloys, nichrome (nickel chrome alloy) wires, stainless steel, kanthal filaments or any material which does not outgas at the desired temperatures, are located around the outside annular wall 23A of the receiver 23 to the annular inner wall 23B of the receiver. is fixed within the furnace. Optionally insulation 911 may wrap at least some of the outside annular wall 23A of the receiver. The receiver 23 may span from the point heated air exits the open top 23 of the furnace to the touches the material in the material chamber 810, then through the flow channel to below the outlet 802. In practice the bottom edge 805 of the optional spacer 804 is configured to fit into the interface 24 to assist with positioning and spacing of the chamber of material 810 in the receiver.

A chassis 935 is a preferred means to space the receiver (duct) in an aligned position with the furnace. The Chassis is configured to can circuit boards, batteries and support connections and illumination components. However, those of ordinary skill in the art will recognize that the chassis may be eliminated in the power and control elements placed in a casing without departing from the scope of the disclosure. The chassis 935 shown has a chassis top 935′ and a chassis bottom 935″ extended radial wall to position it within the body. In some instances, the chassis is below the furnace. In some instances, it fits around the receiver and is placed above the furnace. If placed above the furnace a central core 937 of the chassis fits around the receiver and may be solid, segmented, a series of studs with air gaps or any configuration which allows insertion of the fluid pathway. The battery power supply 50 and the control board(s) which contains the electrical components to manage temperature, adjust power, activate and change the output of the communication illumination, receive instructions from an app, it may support the pulse width modulation sensor inputs and battery charge discharge control. It may contain an 802.11 chip for wireless data exchanges and support wired data connections as well as other user interfaces. The board 87 may be one or more printed circuit board(s) PCB and the like and is also affixed to the chassis 935. Some connection wires from the heating element to the PCB are shown. The other electrical switches and sensors are also connected to the control board(s). The control board(s) are in signal communications with electrical components of the vaporizer, including but not limited to temperature sensor(s), battery, illumination, on/off switch, charging board, display(s), user interface, input/output and applications that may be used to communicate with the control board(s).

FIGS. 13D-13H show aspects of the gasket 1000 and aspects of the furnace which has both an inner surface 901′ and an outer surface 901″. One or both of the surfaces may be coated, anodized, electroplated, laminated and/or otherwise adhered or fixed to another material. Optionally a fluidly connected divider also known as an air permeably element 910 which is generally thin, conductive, and with perforations or holes to allow air passage may be fitted into the open top 902. The permeable element may be a metal disk with drilled or laser etched holes. Depending on the usage and how much heat is to be stored in the fluidly connected divider 910, the fluidly connected divider 910 may be very thin (thousandths of an inch) or thicker. A thicker metal (or conductive) fluidly connected divider 910 will act as a heat sink which can be used to provide radiation and conduction of heat a chamber of material inserted in the open top 902 in addition to the heated convection air flow.

The heating system 40 is heated with power from the battery power supply 50 and the action of the heating is adjusted via the controller 30 which receives sensor data from at least one temperature sensor 34 such as a thermistor or other thermocouple. The temperature sensor(s) 34 may be placed inside the furnace and/or outside the furnace. The control board contains a microprocessor, memory and software which may include look up tables and may have pulse width modulation functionality. The control board processes the sensor data and adjusts power to the heating element to achieve a predetermined or pre-set, or selected temperature.

FIGS. 13E-13H show aspects of the isolation of the RFP (consumable) inserted into the receiver along arrow 888 configured to be held by the band 1015 of a gasket 1000. The insulation member 1010 forms a connection between the furnace and duct (as discussed above) also referred to as a receiver 23. The band 1015 band may be used to one or more of physically and thermally separate the receiver 23 (duct) and the open top of the furnace. When connected to the receiver's distal end 927 the band 1015 reduces the diameter of the passage formed there through to less than the i.d. “D” of the receiver. The band is used to separate the material chamber 810 having a cross sectional maximum diameter less than the i.d. “D” of the receiver, when a user inserts the RFP into the receiver. The material compresses the band 1015 and the uneven portion 1016 and the RFP passes into the receiver through the gasket and is isolated by the insulating 1015 and its uneven portion 1016 wherein the material chamber 810 is positioned remote from the inner wall of the receiver and “air” remains in the areas of the receiver between the exterior of the RFP and the inner wall of the receiver (see FIGS. 13E and 13H).

It will be understood that various aspects or details of the disclosures may be changed combined, or removed without departing from the scope of the invention. It is not exhaustive and does not limit the claimed inventions to the precise form disclosed. Furthermore, the foregoing description is for the purpose of illustration only, and not for the purpose of limitation. Modifications and variations are possible in light of the above description or may be acquired from practicing the invention. The claims and their equivalents define the scope of the invention.

What is claimed is:

1. A vaporizer system for non-combustion vaporization of plant material in a cartridge, the vaporizer system comprising:
a controller;
a power supply in signal communication with the controller;
a heater with at least one heating element affixed cross-sectionally around a portion of an outer wall of a generally tubular receiver having an open end, wherein each heating element is controlled via power allocated by the controller;
at least one temperature sensor in thermal communication with the wall of the receiver and in signal communication with the controller;
a cartridge having an inhalation end and a containment portion configured to fit within the receiver and be in thermal communication with the at least one heating element;
at least tobacco material within the containment portion;
an aliquot of air within the receiver between the inner wall and the cartridge, wherein the receiver is a heat transfer body configured to pass heat from the heater to at least the air within the receiver and the containment portion; and
a fluid pathway configured for air to enter the receiver;
wherein
the controller is configured to receive a temperature sensor input and control. a timing and/or an amount of power applied to activate the at least one heating element and,
the fluid pathway is at least one of an air intake and the open end of the receiver.

2. The vaporizer system of claim 1, further comprising:
multiple conductive regions within the inner wall of the containment portion.

3. The vaporizer system of claim 2, wherein the conductive regions in the containment portion are separated from each other by non-conductive areas of the inner wall of the containment portion.

4. The vaporizer system of claim 1, wherein the plant material includes at least two of hemp, cannabis and tobacco.

5. A method of vaporizing material in a disposable cartridge within a receiver, the method comprising:
placing one or more heating elements affixed cross sectionally as rings around an outside wall of a receiver configured to contain air and receive the disposable cartridge;
placing at least one temperature sensor in thermal communication with at least one of the outside wall and an inner wall of the receiver;
inserting the disposable cartridge inside the receiver, wherein;
the disposable cartridge has an inhalation end extending beyond the receiver and a containment end holding material to vaporize within the receiver;
the receiver is configured as a heat transfer body to transfer heat from the one or more heating elements to at least a portion of the air in the receiver between the disposable cartridge and the inner wall and to the containment end of the disposable cartridge; and
forming signal communications between the one or more heating elements, the at least one temperature sensor and the controller.

6. The method of claim 5, wherein the controller receives temperature sensor input and controls the timing and/or an amount of power applied to activate a heating element, of the one or more heating elements, to heat the air in the receiver and the containment end to cause vaporization, without combustion, of the material.

7. The method of claim 6, wherein the disposable cartridge has at least one thermally conductive region in the containment end.

8. The method of claim 6, wherein the containment end of the disposable cartridge does not burn, or combust at a temperature configured to vaporize plant material after at least one minute of exposure to such temperature.

9. The method of claim 6, wherein the containment portion of the cartridge is configured to not burn, or combust at a temperature configured to vaporize plant material after at least five minutes of exposure to such temperature.

10. The method of claim 5, further comprising one or more indicator, wherein a status of a system is visually communicated, and
the system includes the disposable cartridge and the receiver.

11. The method of 6, wherein the material is at least one of hemp, cannabis and tobacco.

12. The vaporizer system of claim 1, wherein the controller is configured to switch the heating element based on at least one of a time and a temperature.

13. The vaporizer system of claim 12, further comprising at least one thermally conductive region within the inner wall of the containment portion in thermal communication with the inside wall of the receiver, wherein the inside wall is in thermal communication with one or more heating elements.

14. A disposable cartridge configured to fit within a receiver, comprising:
a disposable generally hollow outer wrap having at least one frangible section forming a generally hollow consumable;
said consumable having at least a containment end and an inhalation end;
material containing at least tobacco within the containment end;
at least one frangible section of the outer wrap is configured to bend inward when passing over an actuator; and,
at least one flow through divider placed within the consumable.

15. The disposable cartridge of claim 14, wherein the at least one flow through divider is placed in the inhalation end.

16. The disposable cartridge of claim 14, wherein the at least one flow through divider is placed at the containment end.

17. The disposable cartridge of claim 14, wherein one of the at least one flow through dividers is placed at the containment end and one is placed between the material and the inhalation end.

18. The disposable cartridge of claim 14, further comprising at least one thermally conductive region within the containment end.

19. The disposable cartridge of claim 18, wherein the at least one thermally conductive region is one of metal and foil.
